(19) United States
(12) Patent Application Publication
(10) Pub. No.: US 2011/0087081 A1
(43) Pub. Date: Apr. 14, 2011

(54) PERSONALIZED PHYSIOLOGICAL MONITOR

(76) Inventors: Massi Joe E. Kiani, Laguna Niguel, CA (US); Michael O'Reilly, Dana Point, CA (US)

(21) Appl. No.: 12/849,808

(22) Filed: Aug. 3, 2010

Related U.S. Application Data
(60) Provisional application No. 61/231,011, filed on Aug. 3, 2009.

(51) Int. Cl. A61B 5/00; G06F 19/00; G01N 33/48

(52) U.S. Cl. 600/301; 702/20; 702/19

(57) ABSTRACT

A personalized physiological monitor utilizes an individual genome sequence along with genetic and medical research databases so as to define a person's genetic predisposition to disease, drug reactions and environmental sensitivities so as to enhance the ability of the monitor to determine the physiological status of the person.

### CROSS-REFERENCE TO RELATED APPLICATIONS

The present application claims priority benefit under 35 U.S.C. §119(e) to U.S. Provisional Patent Application Ser. No. 61/231,011, filed Aug. 3, 2009, titled Personalized Physiological Monitor, hereby incorporated by reference herein.

### BACKGROUND OF THE INVENTION

Genes and the environment play a central role in the development and progression of common diseases such as diabetes, cancer, heart disease, stroke, depression and asthma as well as individual responses to pharmacological drugs and medicines. Genes are the instructions that determine our physical being, leading to differences in appearance and differences in how our bodies function. Accordingly, variations in genes affect health risks, i.e. our susceptibility to both common and rare diseases.

An individual genome sequence is a determination of the chemical base pairs that make up the DNA of a single person. The Human Genome Project and a parallel project by Celera Genomics each produced and published a human genome sequence using composite DNA sequences of several individuals. Following that, the International HapMap Project developed a human genome map that describes human genetic variation. Medical treatments have different effects on different people because of genetic variations such as single-nucleotide polymorphisms (SNPs). HapMap focused only on common SNP's, choosing a sample of 269 individuals and selecting several million well-defined SNPs, genotyping the individuals for these SNPs, and publishing the results. Also, there are many genetic disorders annotated in the Online Mendelian Inheritance in Man (OMIM) database of human genes and genetic disorders developed at Johns Hopkins. The Human Genome Project and HapMap allow the exploration of subtle genetic influences on many common disease conditions such as diabetes, asthma, migraine, schizophrenia.

The sciences of pharmacogenetics or pharmacogenomics study the genetic variations that can cause different patients to respond in different ways to the same medication. Advances in understanding the genetic basis of individual drug responses come from the NIH Pharmacogenetics Research Network (PGRN), a nationwide alliance of research groups that has studied genes and medications relevant to a wide range of diseases. Scientists identified more than 1 million genetic variations, many of which may relate to disease risk or drug responses. NextBio, Cupertino, Calif. is one instance of a commercial information provider that enables life science researchers to search, discover, and share knowledge regarding genes, diseases and compounds that are compiled within public and proprietary databases. In addition, organizations such as Illumina, Inc., San Diego, Calif. are offering personal genome sequencing for consumers, including sequencing of an individual's DNA and providing information on SNP variation and other structural characteristics of the genome such as insertions, deletions and rearrangements.

### SUMMARY OF THE INVENTION

Physiological monitoring capabilities are enhanced with information regarding individual genomic sequence variation and medical history in conjunction with medical and genetic research in these areas. Physiological measurements themselves are improved to the extent that such measurements are a function of individual differences in genetics, health and environment. Also, physiological measurements result in tailored monitor outputs, such as alarms, wellness indicators, controls and diagnostics. Such personalized monitoring advantageously allows improved accuracy of measurements and personalized treatment in comparison to a “one size fits all” monitor that is based solely upon a generalized measure of physiological status.

One aspect of a personalized physiological monitor is a system that utilizes an individual genome sequence along with genetic and medical research databases so as to define a person's genetic predisposition to disease, drug reactions and environmental sensitivities. In this manner, the ability of a monitor to determine the physiological status of the person is enhanced. This personalized physiological monitor system utilizes sensor data and personalization data. The sensor data is responsive to a physiological state of a person. The personalization data is responsive to an individual genome sequence for the person so as to indicate predispositions to disease, interactions to drugs and sensitivities to environment. A physiological monitor is responsive to the sensor data and the personalization data so as to generate a physiological status output particularized for the person.

In various embodiments, the personalized physiological monitoring system has databases relating to medical records and medical and genetic research. Personalization data is derived from the databases and the individual genome sequence. The physiological monitor has physiological parameter devices responsive to the sensor data so as to derive physiological parameters. A physiological parameter processor operates on the physiological parameters and the personalization data so as to derive the physiological status output. The physiological status output has a wellness indicator that provides a tailored measure of the health of the person according to the personalization data. The physiological parameter processor has a particularized metric based upon the personalization data. A test of the particularized metric is based upon the personalization data. The physiological monitor may comprise plug-ins corresponding to the physiological parameter devices. A display graphic indicates which plug-ins are to be installed into the physiological monitor. The display graphic is responsive to the personalization data.

In other various embodiments, the physiological monitor has a docking station and a shuttle station that removably attaches to the docking station. The docking station is capable of generating a first set of physiological parameters and the shuttle station is capable of generating a second set of physiological parameters. The personalization data is communicated to the docking station, and at least a portion of the personalization data is communicated from the docking station to the shuttle station so that the shuttle station provides a particularized shuttle station output according to the personalization data portion regardless of separation from the docking station. The physiological status output may further comprise a personalization indicator that displays a measure of the extent that the wellness indicator is based upon the personalization data.

Another aspect of a personalized physiological monitor comprises determining an individual genome sequence of a person, specifying individual differences regarding at least some of disease predisposition, drug reaction and environmental sensitivity based upon the individual genome sequence, and personalizing the response of the physiological monitor to the person based upon the individual differences. Various embodiments include creating a personalization database that reflects the individual differences and tailoring the response of a physiological monitor to the person according to the personalization database. Creating may comprise accessing a genetic research database so as to determine the relationship between specific genes in the individual genome sequence and the individual differences.

Various other embodiments further include outputting a wellness indicator that provides a tailored prediction of health based upon the individual differences and providing a personalization indicator that relates the extent the wellness indicator is responsive to the individual differences. Additional embodiments include configuring the monitor to make particularized physiological measurements on the person based upon the individual differences and downloading personalization data from a docking station to a shuttle station before removing the shuttle station from the docking station.

A further aspect of a personalized physiological monitor comprises sensors in communications with a person and generating sensor outputs. A personalization data input is responsive to an individual genome sequence. Physiological parameter devices are responsive to the sensor inputs so as to generate physiological parameters. A parameter processor is responsive to the physiological parameters and the personalization data so as to generate a physiological status output. In various embodiments, a personalization indicator is responsive to the relative contribution of the personalization data to the physiological status output. A combination of the physiological parameters is defined according to the personalization data so as to generate a metric that is responsive to the individual genome sequence. A rule is applied to the metric according to the personalization data so as to generate a test result. A wellness indicator is output according to the test result and the personalization data. The personalization data is modified according to a genetic research database.

### BRIEF DESCRIPTION OF THE DRAWINGS

FIG. 1 is a general block diagram of a personalized physiological monitoring system;

FIG. 2 is a general block diagram of a personalized physiological monitor;

FIG. 3 is a general block diagram of a physiological parameter processor;

FIGS. 4A-D are front, side docked, side partially undocked and side fully undocked views of a personalized physiological monitor embodiment; and

FIG. 5 is an illustration of a physiological monitor display embodiment.

### DETAILED DESCRIPTION OF THE PREFERRED EMBODIMENTS

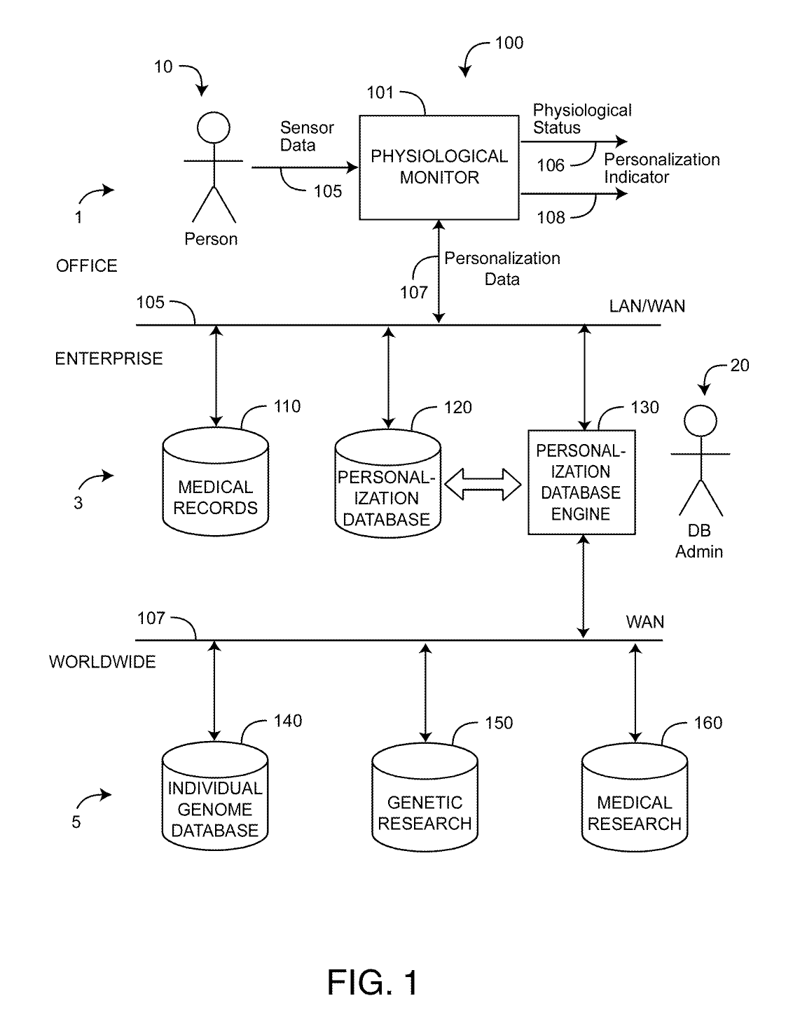

FIG. 1 illustrates a multi-tier personalized physiological monitoring system 100 having office 1, enterprise 3 and worldwide 5 levels. The office 1 level denotes a location where a patient is evaluated and treated for various medical conditions, such as a medical office, a clinic or urgent care center. The enterprise 3 level denotes a hospital or an administrative center for one or more medical providers. The worldwide 5 level denotes all commercial, scientific and scholastic resources available for medical and genetic-related knowledge. At the enterprise level 3, a database administrator 20 or related personnel accesses one or more individual genome 140, genetic research 150 and medical research 160 databases for information that can be compiled and organized into a personalization database 120.

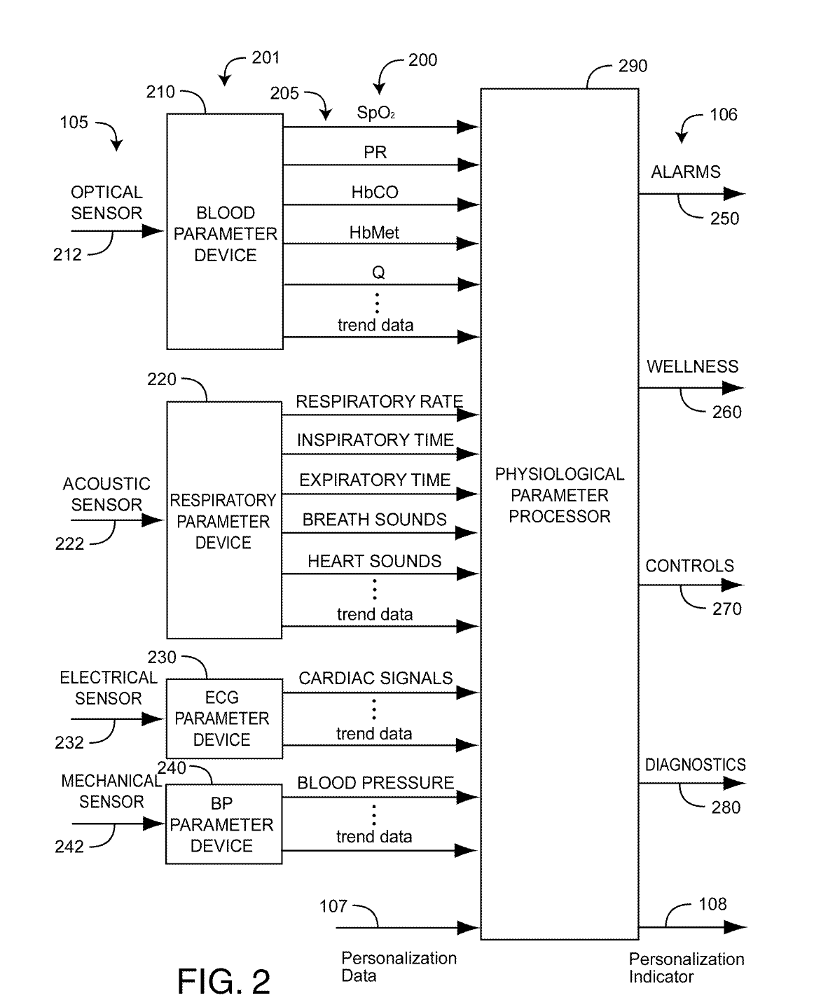

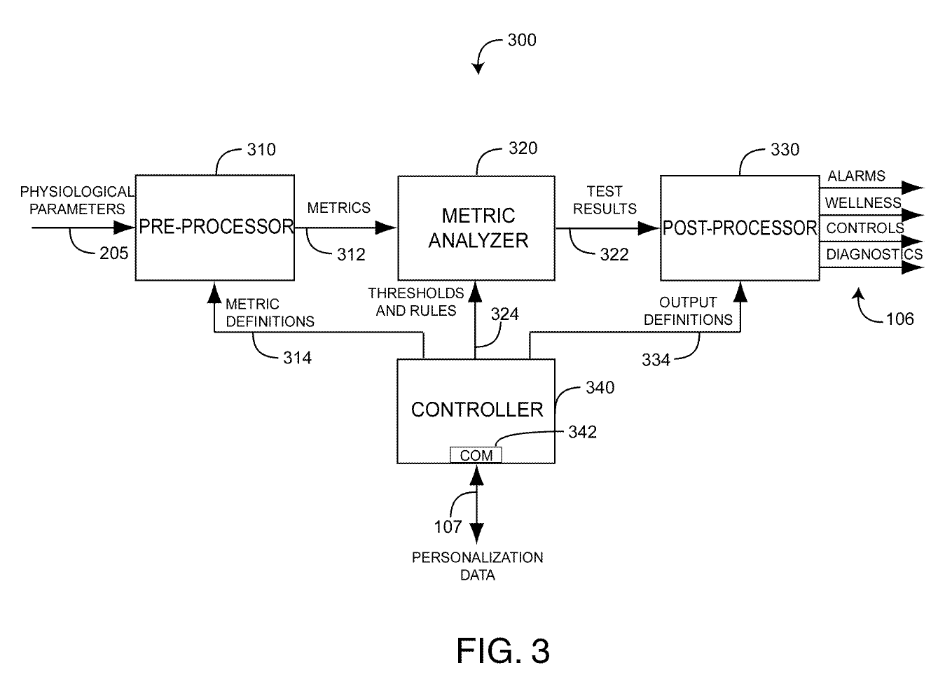

As a few examples, the individual genome database 140 includes results from sequencing the DNA of specific individuals. Genetic research 150 includes information regarding the human genome sequence and genetic variations, and the relationship between such genetic variations and associated environmental factors and corresponding diseases and treatments. Medical research 160 includes non-genetic information relating to the diagnosis and treatment of injury, illness and disease. The database administrator 20 also accesses individual medical records 110 of patients for current and historical information regarding family, employment, habits, physical health and medical conditions that may provide further information regarding individual genetics, environmental exposure, health, diseases and treatment. The database administrator 20, or other personnel, utilizes a database engine 130 to generate a personalization database 120, which contains personalization data 103 that can be accessed by a personalized physiological monitor 101 at the medical provider level 1, as described in further detail with respect to FIGS. 2-3, below. In an alternative embodiment, the personalization database 120 is compiled or updated, at least partially, by crawlers, spiders, bots or other automatic data gathering techniques applied to the databases 140, 150, 160.

FIG. 2 illustrates a personalized physiological monitor 200 embodiment having any of various sensors 105 and corresponding physiological measurement devices 201. The devices 201 may be standalone monitors, plug-ins or modules, to name a few, which measure single parameters or parameter types or multi-parameter measurement devices. As such, devices may be separate from or incorporated in whole or part within a personalized parameter processor 290. The physiological measurement devices 201 input sensor data 105 and generate corresponding physiological parameters 205. The personalized parameter processor 290, which may comprise an expert system, a neural-network or a logic circuit, as examples, inputs one or more parameters 205 from one or more physiological measurement devices 201 and generates physiological status outputs 106.

As shown in FIG. 2, physiological measurement devices 201 may include a blood parameter device 210, a respiratory parameter device 220, an electrocardiogram (ECG) parameter device 230 and a blood pressure (BP) parameter device 240, as a few examples. The corresponding sensors 105 may be, for instance, invasive or noninvasive optical 212, acoustic 222, electrical 232 or mechanical 242 sensors. Blood parameters may include oxygen saturation (SpO₂), perfusion index (P1), pulse rate (PR), pleth variability index (PVI), HbCO, HbMet and other abnormal hemoglobin measurements, various signal quality and/or data confidence indicators (Q) and trend data, to name a few. Other physiological measurement devices not shown are also included within the scope of this embodiment, such as a capnometer and devices to measure blood glucose and temperature or any other invasive or noninvasive physiological monitoring devices or the like.

The physiological parameters 205 are processed alone or in combination to generate one or more physiological status outputs 106 comprising alarms 250, wellness indicators 260, controls 270 and diagnostics 280. Alarms 250 may be used to alert medical personnel to a deteriorating condition in a person under their care. Wellness indicators 260 may provide a general measure of a person's overall medical condition. Controls 270 may be used to affect the operation of a medical-related device. Diagnostics 280 may be used to assist medical personnel in determining specific causes of a person's medical condition.

Also shown in FIG. 2, personalization data 107 utilizes an individual genome sequence along with genetic and medical research databases so as to define a person's genetic predisposition to disease, possible physiological abnormalities or weaknesses, potential negative reactions drugs or drugs that may be ineffective and sensitivities to environment conditions, as a few examples. Personalization data 107 therefore enhances the ability of a monitor 200 to determine a person's physiological status 106.

Further shown in FIG. 2, a personalization indicator 108 provides a measure of the extent that individual genes and medical history impact the physiological status outputs 106. For example, the personalization indicator 108 may be a number or equivalent graphic that varies between “0” and “100.” In this example “0” indicates that the physiological status 106 is not personalized but rather is representative of what the measured parameters 205 indicates for the population at large. “100” indicates that the physiological status 106 is strongly dependent on or otherwise substantially affected by a person's individual medical history and genetic predispositions. Advantageously, the personalization indicator 108 notifies a caregiver, for example, that a combination of physiological parameters 205 having otherwise normal range values may indicate a potentially serious condition for this particular person, as reflected by alarms 250, wellness indicators 260 and other status outputs 106. In other embodiments, the personalization indicator 108 may range from a simple on/off indicator indicating at least some personalization to a detailed graphic or description specifying the contribution of one or more genes or SNPs.

FIG. 3 illustrates a physiological parameter processor 300 embodiment responsive to physiological parameters 205 so as to generate a physiological status 106. In an embodiment, the parameter processor 300 has a pre-processor 310, a metric analyzer 320, a post-processor 330 and a controller 340. The pre-processor 310 has parameter data inputs 205 derived from one or more devices 201 (FIG. 2). The pre-processor 310 generates metrics 312 that may include, as examples, pass-thru parameters and sensor waveforms, multiple-channel derived parameters, such as a rising pulse rate and a falling blood pressure and cross-channel comparisons, such as the highest signal quality pulse rate derived from an optical, an acoustic, an electrical and a mechanical sensor.

As shown in FIG. 3, the metric analyzer 320 is configured to test metrics 312 and communicate the test results 322 to the post-processor 330 based upon various rules and thresholds 324 applied to the metrics 312. As an example, the metric analyzer 320 may communicate to the post-processor 330 when a parameter measurement increases faster than a predetermined rate, e.g. a trend metric exceeds a predetermined trend threshold.

Also shown in FIG. 3, the post processor 330 inputs the test results 322 and generates alarm, wellness, control and diagnostic outputs 106 based upon output definitions 334. For example, if the test is whether a trend metric exceeds a trend threshold, then the output definition corresponding to that test result may be to trigger an audible alarm.

Further shown in FIG. 3, the controller 340 has an external communications port 342 that provides network (e.g. LAN) communications to an external device, such as the personalization database 120 (FIG. 1) and corresponding personalization data 107. Based upon the patient personalized-information, the controller 340 transmits thresholds and rules 324 to the metric analyzer 320. The controller 340 may also provide metric definitions 314 to the pre-processor 310 and define outputs 334 for the post-processor 330. In a particularly advantageous embodiment, the controller 340 configures the physiological parameter processor to define, via the metrics definitions 314 and thresholds/rules 324, metrics 312, tests 322 and outputs 106 particularized for a specific person based upon some aspect of their individual genome sequence and/or medical history. Accordingly, the personalized physiological monitor 100, 200 dynamically configures itself or otherwise adapts itself to monitor for a particular person's genetic predisposition to disease, possible physiological abnormalities or weaknesses, potential negative reactions drugs or drugs ineffectiveness and sensitivities to environmental conditions, such as allergies, dust, molds and chemicals to name a few.

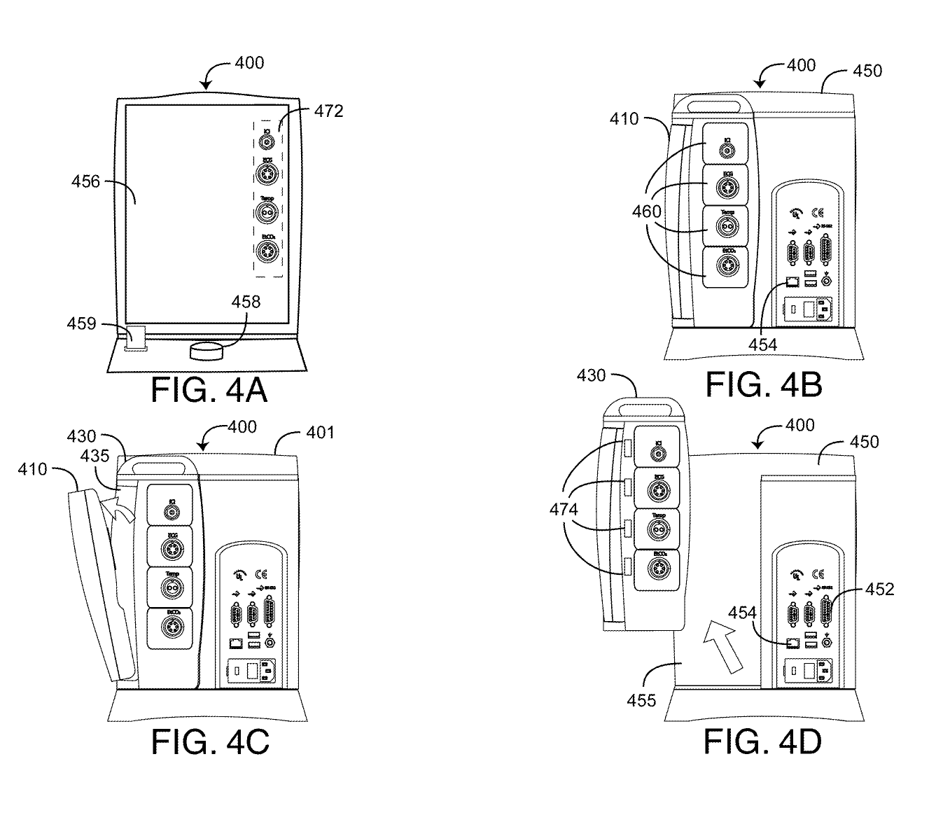

FIGS. 4A-D illustrate a personalized physiological monitor 400 embodiment having a handheld monitor 410, a shuttle station 430 and a docking station 450. The docking station 450 has a shuttle port 455 that allows the shuttle station 430 to dock. The shuttle station 430 has a handheld port 435 that allows the handheld monitor 410 to dock. Accordingly, the modular patient monitor 400 has three-in-one functionality including a handheld 410, a handheld 410 docked into a shuttle station 430 as a handheld/shuttle 440 and a handheld/shuttle 440 docked into a docking station 450. When docked, the three modules of handheld 410, shuttle 430 and docking station 450 function as one unit. The docking station 450 charges the handheld 410 and shuttle 430, provides a larger screen 456 and controls, such as a trim knob 458, allows wireless, hardwired and Internet communications 452 and provides connectivity to various external devices. The shuttle 430 also has plug-in modules 460 for expanded parameter functionality. In an embodiment, the handheld monitor 410 incorporates blood parameter devices for measuring blood parameters including SpO₂, PR, HbCO, HbMet and Hbt to name a few. In an embodiment, the shuttle station 430 incorporates non-blood parameters, such as intelligent cuff inflation (101), end-tidal CO₂ (EtCO₂), acoustic respiration rate (ARR), patient body temperature (Temp) and ECG, to name a few. The docking station 450 has one or more network connectors 454 providing access to personalization data 107 (FIG. 2), as described above.

In an advantageous embodiment, the personalized physiological monitor 400 determines a monitor configuration particularly adapted to the personalization data 107 and indicates the necessary plug-ins 460, sensors and connections to achieve that configuration. As an example, a set of needed plugs-in may be indicated on the monitor display 456 as graphics 472 or descriptions. As another example, LEDs 474 may illuminate according to a predetermined color code to indicate needed plug-ins and/or sensors.

Further shown in FIGS. 4A-D, personalization data 107 (FIG. 2) is advantageously downloaded in whole or in part from the docking station 450 to the satellite station 430 and from satellite station 430 to the handheld 410 so that the satellite station 430 and the handheld 410 provide personalized monitoring after detachment from the docking station 450 or the satellite station 430.

In other embodiments, a personalized physiological monitor dynamically reconfigures one or more of sensors, sensor configurations, physiological parameter devices and displays, to name a few, in response to the personalization data. Although described above as utilizing local or wide-area networks and databases to provide personalization data, in other embodiments personalization data may be compiled on a plug-in memory card 459 or other portable memory device. A physiological monitor having a docking station, shutter station and handheld monitor is described in U.S. patent application Ser. No. 11/903,746 filed Sep. 24, 2007 and titled Modular Patient Monitor, assigned to Masimo Corporation, Irvine Calif. (Masimo) and hereby incorporated by reference herein.

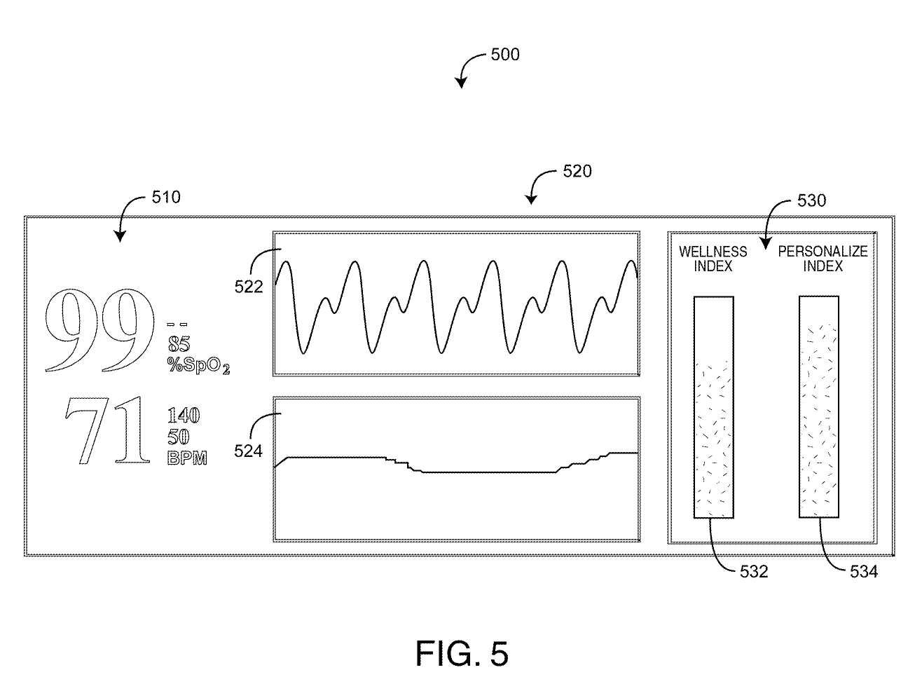

FIG. 5 illustrates a personalized physiological monitor display 500 having parameter readouts 510, parameter graphics 520 and status outputs 530. In an advantageous embodiment, one or more of the readouts 510, graphics 520 and status 530 are responsive to personalization data 107 (FIG. 2). In an advantageous embodiment, personalized outputs and non-personalized outputs are displayed for comparison. For example, parameter graphics 520 may include “standard” parameter displays 522, such as a plethysmograph and a personalized parameter display 524, such as a trend of multiple parameters combined to specifically detect a physiological condition indicated by one or more SNP's of an individual genome sequence. As another example, the status outputs 530 may include a wellness index 532 display, as described above, and a personalized index 534 display indicating the extent that the wellness index incorporates or is otherwise impacted by personalized parameter measurements. Wellness outputs and displays are described in U.S. patent application Ser. No. 11/963,640 filed Dec. 21, 2007 and titled Physiological Parameter System, assigned to Masimo and hereby incorporated by reference herein.

A personalized physiological monitor has been disclosed in detail in connection with various embodiments. These embodiments are disclosed by way of examples only and are not to be construed as limiting the scope of the claims that follow. One of ordinary skill in art will appreciate many variations and modifications.

What is claimed is:

1. A personalized physiological monitoring system utilizes an individual genome sequence along with genetic and medical research databases so as to define a person's genetic predisposition to disease, drug reactions and environmental sensitivities and therefore enhance the ability of a monitor to determine the physiological status of the person, the personalized physiological monitoring system comprising:
sensor data responsive to a physiological state of a person;
personalization data responsive to an individual genome sequence for the person so as to indicate predispositions to disease, interactions to drugs and sensitivities to environment; and
a physiological monitor responsive to the sensor data and personalization data so as to generate a physiological status output particularized for the person.

2. The personalized physiological monitoring system according to claim 1 further comprising:
a plurality of databases relating to medical records and medical and genetic research; and
personalization data derived from the databases and the individual genome sequence.

3. The personalized physiological monitoring system according to claim 2 wherein the physiological monitor comprises:
a plurality of physiological parameter devices responsive to the sensor data so as to derive a plurality of physiological parameters;
a physiological parameter processor that operates on the physiological parameters and the personalization data so as to derive the physiological status output; and
the physiological status output comprising a wellness indicator that provides a tailored measure of the health of the person according to the personalization data.

4. The personalized physiological monitoring system according to claim 3 wherein the physiological parameter processor comprises:
a particularized metric based upon the personalization data; and
a test of the particularized metric based upon the personalization data.

5. The personalized physiological monitoring system according to claim 4 wherein the physiological monitor comprises:
a plurality of plug-ins corresponding to the physiological parameter devices;
a display graphic that indicates the plug-ins are to be installed into the physiological monitor; and
the display graphic responsive to the personalization data.

6. The personalized physiological monitoring system according to claim 5 wherein the physiological monitor comprises:
a docking station;
a shuttle station that removably attaches to the docking station;
the docking station capable of generating a first set of physiological parameters;
the shuttle station capable of generating a second set of physiological parameters;
the personalization data communicated to the docking station; and
at least a portion of the personalization data communicated from the docking station to the shuttle station so that the shuttle station provides a particularized shuttle station output according to the personalization data portion regardless of separation from the docking station.

7. The personalized physiological monitoring system according to claim 6 wherein the physiological status output further comprises a personalization indicator that displays a measure of the extent that the wellness indicator is based upon the personalization data.

8. A personalized physiological monitoring method comprising:
determining an individual genome sequence of a person;
specifying a plurality of individual differences regarding at least some of disease predisposition, drug reaction and environmental sensitivity based upon the individual genome sequence; and
personalizing the response of a physiological monitor to the person based upon the individual differences.

9. The personalized physiological monitoring method according to claim 8 further comprising:
creating a personalization database that reflects the individual differences; and
tailoring the response of a physiological monitor to the person according to the personalization database.

10. The personalized physiological monitoring method according to claim 9 wherein creating comprises accessing a genetic research database so as to determine the relationship between specific genes in the individual genome sequence and the individual differences.

11. The personalized physiological monitoring method according to claim 10 further comprising outputting a wellness indicator that provides a tailored prediction of health based upon the individual differences.

12. The personalized physiological monitoring method according to claim 11 further comprising providing a personalization indicator that relates the extent the wellness indicator is responsive to the individual differences.

13. The personalized physiological monitoring method according to claim 12 further comprising configuring the monitor to make particularized physiological measurements on the person based upon the individual differences.

14. The personalized physiological monitoring method according to claim 13 further comprising downloading personalization data from a docking station to a shuttle station before removing the shuttle station from the docking station.

15. A personalized physiological monitor comprising:
a plurality of sensors in communications with a person and generating sensor outputs;
a personalization data input responsive to an individual genome sequence;
a plurality of physiological parameter devices responsive to the sensor inputs so as to generate a plurality of physiological parameters; and
a parameter processor responsive to the physiological parameters and the personalization data so as to generate a physiological status output.

16. The personalized physiological monitor according to claim 15 further comprising a personalization indicator responsive to the relative contribution of the personalization data to the physiological status output.

17. The personalized physiological monitor according to claim 16 further comprising a combination of the physiological parameters defined according to the personalization data so as to generate a metric that is responsive to the individual genome sequence.

18. The personalized physiological monitor according to claim 17 further comprising a rule applied to the metric according to the personalization data so as to generate a test result.

19. A personalized physiological monitor according to claim 18 further comprising a wellness indicator output according to the test result and the personalization data.

20. A personalized physiological monitor according to claim 19 further comprising the personalization data modified according to a genetic research database.
